(19) United States
(12) Patent Application Publication
(10) Pub. No.: US 2008/0102480 A1
(43) Pub. Date: May 1, 2008

(54) Method and Apparatus of Enzyme-Link Immunosorbent Assay

(75) Inventor: Chien-Chen Chen, Taipei (TW)

Correspondence Address: WPAT, PC, 7225 BEVERLY ST., ANNANDALE, VA, 22003, US

(73) Assignee: EBIOLOGIC TECHNOLOGY CO., LTD., Taipei (TW)

(21) Appl. No.: 11/555,684

(22) Filed: Nov. 1, 2006

(51) Int. Cl. G01N 33/53

(52) U.S. Cl. 435/7.92

(57) ABSTRACT

An improved method of enzyme-link immunosorbent assay (ELISA) is disclosed, which can be adapted to be implemented by an analytic apparatus having a testing platform and a conducting platform, being arranged separate from the testing platform, and comprises steps of: applying a specific agent upon a reaction zone of the testing platform; dripping a testing antibody onto the testing platform for enabling the testing antibody to be bound by the specific antigen; dripping a cleaning fluid onto the testing platform for washing out any excess testing antibody not being bound by the specific antigen; dripping a bio-reagent onto the testing platform whereas the bio-reagent is capable of binding with the specific antigen and the testing antibody; attaching a side of the conducting platform, having conducting circuit formed thereon, to the reaction zone of the testing platform while connecting an extending end of the conducting platform to an evaluation instrument; and dripping an enzyme onto the reaction zone while enabling the evaluation instrument to generate a value of potential representing the reaction result of the enzyme.

### FIELD OF THE INVENTION

The present invention relates to an improved method of enzyme-link immunosorbent assay (ELISA), which can be adapted to be implemented by an analytic apparatus having a testing platform and a conducting platform, being arranged separate from the testing platform, such that, by the aforesaid apparatus, the conducting circuit of the conducting platform will not contact with the reaction zone of the testing platform while an antigen, an antibody, a bio-reagent and an enzyme are reacting in the reaction zone, and thus the conducting circuit will not be contaminated by the reactant of the ELISA method for enabling an evaluation instrument, connected to the conducting platform, to be able to conduct an accurate measurement for acquiring an accurate value of potential corresponding thereto as an enzyme is dripped into the reaction zone to react with the reactant.

### BACKGROUND OF THE INVENTION

In bioscience industry, there are many methods currently available for protein test, such as SDS gel electrophoresis, Western bolt, Immunoprecipitation, Enzyme-link immunosorbent Assay (ELISA), etc. Among which, the ELISA method is advantageous in that it is simple in testing procedure and low cost, so that it is massively applied in commercial protein tests.

ELISA is a simple and high sensitive method of analysis that allows for simultaneous and rapid quantification of a large amount of samples, which is based on the specific recognition of the target compound (antigen/analyte) by antibodies which bind to the compound. The antibody-antigen complex is detected and measured with the aid of an enzyme-labeled antibody or antigen. Upon addition of a non-colored reagent, the enzyme produces a color intensity where the color reaction is directly or inversely proportional to the concentration of the analyte in the sample. A conventional ELISA method is a biochemical technique used mainly in immunology to detect the presence of an antibody or an antigen in a sample. It utilizes two antibodies, one of which is specific to the antigen and the other of which is coupled to an enzyme. This second antibody gives the assay its “enzyme-linked” name, and will cause a chromogenic or fluorogenic substrate to produce a signal. Therefore, the ELISA can be performed to evaluate the presence of the specific antigen in a sample, and thus it is a useful tool both for determining concentration and the presence of antigen.

However, although the aforesaid conventional ELISA method is capable of detecting the presence of a specific antigen, i.e. protein, the qualification thereof can not be determined thereby such that the conventional ELISA method is usually considered to be a rough test by which no further analysis about the specific antigen can be acquired.

Therefore, it is in need of an improved ELISA method capable of quantitate the specific antigen accurately as it is detected thereby and thus presenting the quantification measurement with a potential value.

### SUMMARY OF THE INVENTION

In view of the disadvantages of prior art, the primary object of the present invention is to provide an improved method of enzyme-link immunosorbent assay (ELISA), which can be adapted to be implemented by an analytic apparatus having a testing platform and a conducting platform, being arranged separate from the testing platform, such that, by the aforesaid apparatus, the conducting circuit of the conducting platform will not contact with the reaction zone of the testing platform while an antigen, an antibody, a bio-reagent and an enzyme are reacting in the reaction zone, and thus the conducting circuit will not be contaminated by the reactant of the ELISA method for enabling an evaluation instrument, connected to the conducting platform, to be able to conduct an accurate measurement for acquiring an accurate value of potential corresponding thereto as an enzyme is dripped into the reaction zone to react with the reactant.

It is another object of the invention to provide an ELISA testing platform having a diversion hole, arranged at the circumference of a reaction zone of the testing platform, by which excess testing antibody can be filtered and drained to an area underneath the reaction zone where it is absorbed by a water absorbing polymer for ensuring all the unbound testing antibody to be removed from the reaction zone.

To achieve the above objects, the present invention provides an improved method of enzyme-link immunosorbent assay (ELISA), adapted to be implemented by an analytic apparatus having a testing platform and a conducting platform, being arranged separate from the testing platform, and comprises steps of:
applying a specific agent upon a reaction zone of the testing platform;
dripping a testing antibody onto the testing platform for enabling the testing antibody to be bound by the specific antigen;
dripping a cleaning fluid onto the testing platform for washing out any excess testing antibody not being bound by the specific antigen;
dripping a bio-reagent onto the testing platform whereas the bio-reagent is capable of binding with the specific antigen and the testing antibody;
attaching a side of the conducting platform, having conducting circuit formed thereon, to the reaction zone of the testing platform while connecting an extending end of the conducting platform to an evaluation instrument; and
dripping an enzyme onto the reaction zone while enabling the evaluation instrument to generate a value of potential representing the reaction result of the enzyme

In addition, the present invention provide an ELISA analytic apparatus, which comprises:
a testing platform, having a reaction zone arranged thereon and used for enabling a specific antigen, a testing antibody, a bio-reagent and an enzyme to react with each other therein;
a conducting platform with an extending end, have a conducting circuit arranged at a side thereof while the side with the conducting circuit is adapted for attaching to the reaction zone of the testing platform; and
an evaluation instrument, connected to the extending end of the conducting platform for enabling the same to conduct an accurate measurement upon the reaction result of the reaction zone and thus acquiring a value of potential corresponding thereto.

Other aspects and advantages of the present invention will become apparent from the following detailed description, taken in conjunction with the accompanying drawings, illustrating by way of example the principles of the present invention.

### BRIEF DESCRIPTION OF THE DRAWINGS

FIG. 1 is a top view of a conducting platform of an ELISA analytic apparatus according to the present invention.

FIG. 2 is a bottom view of a conducting platform of an ELISA analytic apparatus according to the present invention.

FIG. 3 is a cross-sectional view of a testing platform of an ELISA analytic apparatus according to the present invention.

FIG. 4 is a three-dimensional diagram showing an LISA analytic apparatus according to the present invention, whereas the testing platform is attached to the conducting platform.

FIG. 5 is a schematic diagram depicting the reaction performed on a testing platform of an ELISA analytic apparatus according to the present invention.

FIG. 6 is a flow chart depicting steps of an improved method of enzyme-link immunosorbent assay according to the present invention.

### DESCRIPTION OF THE PREFERRED EMBODIMENT

For your esteemed members of reviewing committee to further understand and recognize the fulfilled functions and structural characteristics of the invention, several preferable embodiments cooperating with detailed description are presented as the follows.

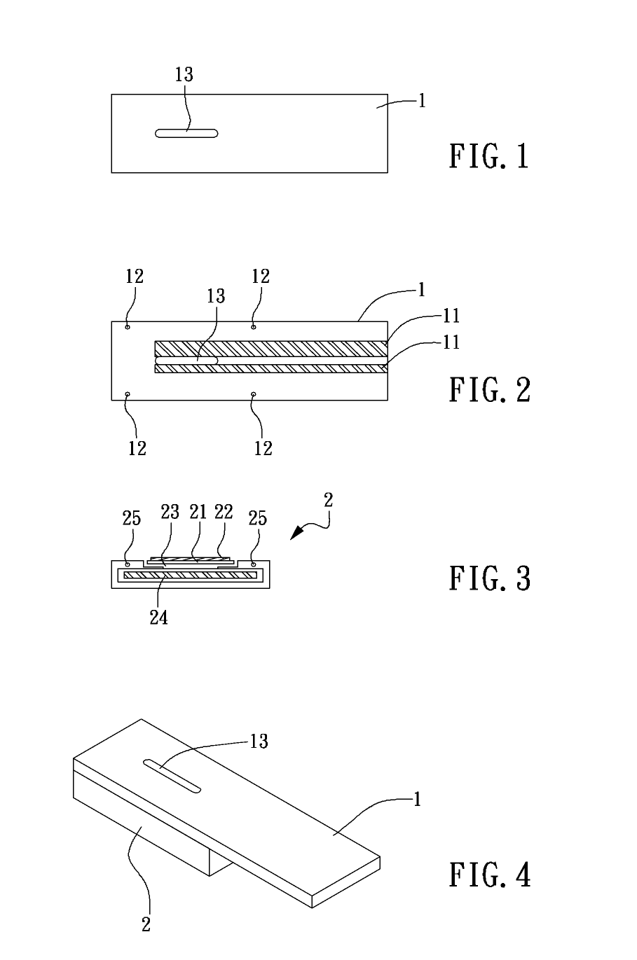

Please refer to FIG. 1 and FIG. 2, which are is a top view and a bottom view of a conducting platform of an ELISA analytic apparatus according to the present invention. As seen, there are two conducting circuits 11 being arranged at a side of the conducting platform 1 that one is thicker then the other while arranging an enzyme-absorption hole 13 to be formed at a specific position between the two. In this preferred embodiment, the enzyme-absorption hole 13 is formed at the center between the two conducting circuits 11. In addition, a plurality of bumps 12, as the four bumps illustrated in this embodiment, are arranged on the conducting platform at positions in the vicinity of the two conducting circuits 11 while each being arranged at a position corresponding to a concave 25 formed on the testing platform 2 for adapting the conducting platform 1 to attached upon the testing platform 2 securely and firmly. In another preferred embodiment, a plurality of wedges are arranged on the conducting platform 1, each being arranged at a position corresponding to a recess formed on the testing platform 2 for adapting the conducting platform 1 to attached upon the testing platform 2 as each wedge is pushed to jammed inside its corresponding recess. Moreover, the adhesion of the conducting platform 1 and the testing platform can be achieved by the use of an adhesive, that is arranged on at least one object selected from the group consisting of the testing platform 2 and the conducting platform 1.

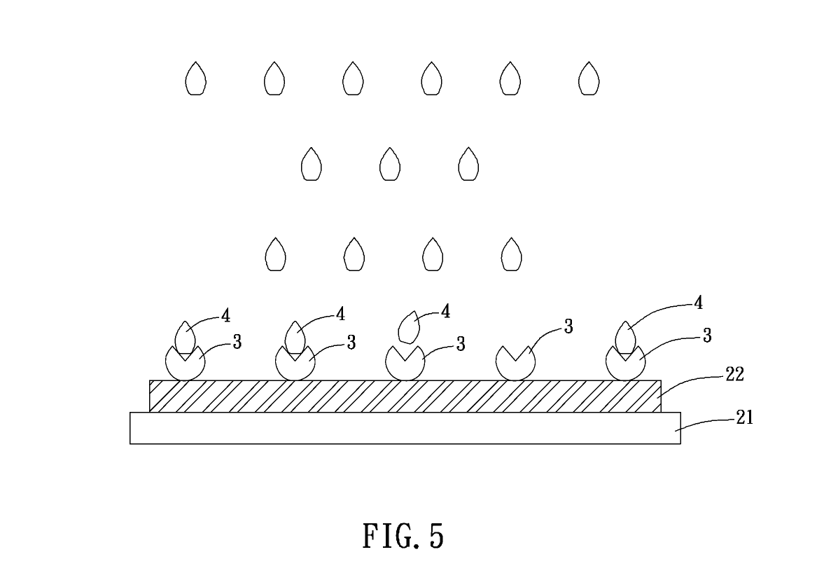

Please refer to FIG. 3 and FIG. 5, which are respective a cross-sectional view of a testing platform of an ELISA analytic apparatus and a schematic diagram depicting the reaction performed on a testing platform of an ELISA analytic apparatus according to the present invention. As seen in FIG. 3 and FIG. 5, there is a reaction zone 21 disposed on a testing platform 2 that is provided for enabling a specific antigen 3, a testing antibody 4, a bio-reagent and an enzyme to react therein. Moreover, a thin film 22 is arranged at the reaction zone 21, which can be made of nitrocellulose or paper; and a diversion hole 23 is arranged at the center of the reaction zone 21 of the testing platform 2 that is used for filtering/draining the excess unbound testing antibody 4 to an area underneath the reaction zone 21 as the thin film 22 is placed covering the diversion hole 23. In addition, a water absorbing polymer 24 is arranged at the area underneath the reaction zone 21 to be used for absorbing the drained excess antibody 4. In a preferred aspect, the water absorbing polymer 24 can be a sponge. As the testing platform 2 and the conducting platform 1 are joined by the clasping of the four bumps 12 of the conducting platform 1 upon the four concaves 25 of the testing platform 2, the conducting circuits 11 can be securely and firmly contact with the reaction zone 21 such that a accurate and quantitative measurement can be achieved.

Please refer to FIG. 4, which is a three-dimensional diagram showing an LISA analytic apparatus according to the present invention, whereas the testing platform is attached to the conducting platform. In FIG. 5, as the conducting platform 1 is attached to the testing platform 2, the extending end of the conducting platform 1 is connected to a testing end of an evaluation instrument for enabling the same to conduct an accurate measurement upon the reaction result of the reaction zone 21 and thus acquiring a value of potential corresponding thereto. It is noted that there are a variety of evaluation instruments that can be used with respect to the kind of protein (antigen) intended to be detected and measured, such as food genes, antibiotics, prisons, insecticide, HIV virus, SARS virus, and so on.

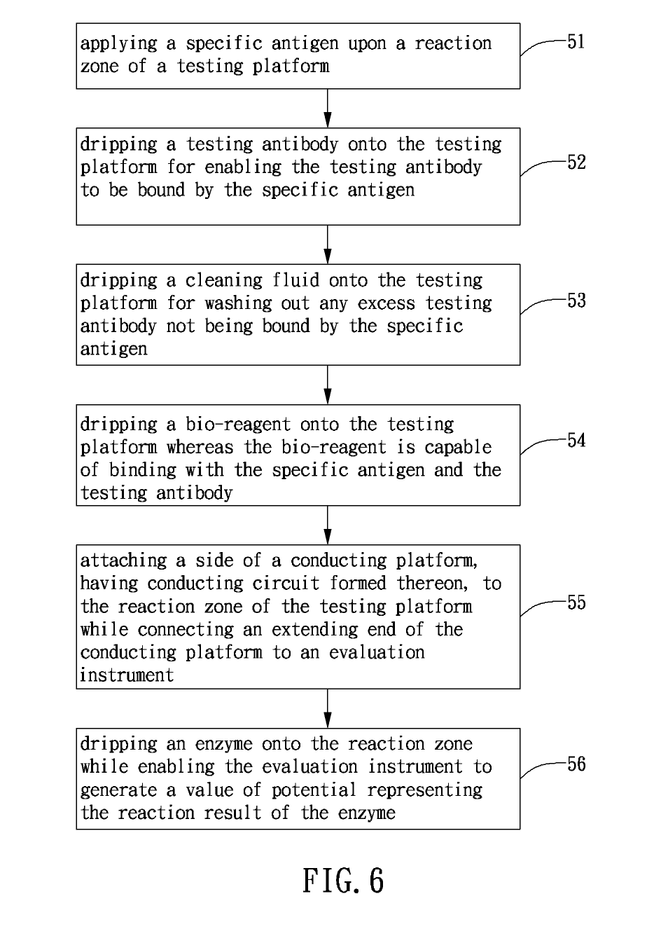

Please refer to FIG. 6, which is a flow chart depicting steps of an improved method of enzyme-link immunosorbent assay according to the present invention. The referring improved method of enzyme-link immunosorbent assay, adapted to be implemented by an analytic apparatus as those shown in FIG. 1 to FIG. 5, comprises steps of:
51 applying a specific agent upon a reaction zone of the testing platform;
52 dripping a testing antibody onto the testing platform for enabling the testing antibody to be bound by the specific antigen;
53 dripping a cleaning fluid onto the testing platform for washing out any excess testing antibody not being bound by the specific antigen;
54 dripping a bio-reagent onto the testing platform whereas the bio-reagent is capable of binding with the specific antigen and the testing antibody;
55 attaching a side of the conducting platform, having conducting circuit formed thereon, to the reaction zone of the testing platform while connecting an extending end of the conducting platform to an evaluation instrument; and
56 dripping an enzyme onto the reaction zone while enabling the evaluation instrument to generate a value of potential representing the reaction result of the enzyme.

From the description of FIG. 1 to FIG. 6, It is clear that the present invention relates to an improved method of enzyme-link immunosorbent assay (ELISA), which can be adapted to be implemented by an analytic apparatus having a testing platform and a conducting platform, being arranged separate from the testing platform, such that, by the aforesaid apparatus, the conducting circuit of the conducting platform will not contact with the reaction zone of the testing platform while an antigen, an antibody, a bio-reagent and an enzyme are reacting in the reaction zone, and thus the conducting circuit will not be contaminated by the reactant of the ELISA method for enabling an evaluation instrument, connected to the conducting platform, to be able to conduct an accurate measurement for acquiring an accurate value of potential corresponding thereto as an enzyme is dripped into the reaction zone to react with the reactant. Moreover, a diversion hole is arranged at the circumference of the reaction zone for draining/filtering excess unbound testing antibody to an area underneath the reaction zone where it is absorbed by a water absorbing polymer, by which the step of dripping a cleaning fluid onto the testing platform for washing out any excess testing antibody not being bound by the specific antigen is ensured to have a preferred effect.

While the preferred embodiment of the invention has been set forth for the purpose of disclosure, modifications of the disclosed embodiment of the invention as well as other embodiments thereof may occur to those skilled in the art. Accordingly, the appended claims are intended to cover all embodiments which do not depart from the spirit and scope of the invention.

What is claimed is:

1. An improved method of enzyme-link immunosorbent assay, adapted to be implemented by an analytic apparatus having a testing platform and a conducting platform, being arranged separate from the testing platform, comprising steps of:
applying a specific agent upon a reaction zone of the testing platform;
dripping a testing antibody onto the testing platform for enabling the testing antibody to be bound by the specific antigen;
dripping a cleaning fluid onto the testing platform for washing out any excess testing antibody not being bound by the specific antigen;
dripping a bio-reagent onto the testing platform whereas the bio-reagent is capable of binding with the specific antigen and the testing antibody;
attaching a side of the conducting platform, having conducting circuit formed thereon, to the reaction zone of the testing platform while connecting an extending end of the conducting platform to an evaluation instrument; and
dripping an enzyme onto the reaction zone while enabling the evaluation instrument to generate a value of potential representing the reaction result of the enzyme.

2. The method of claim 1, wherein a thin film is arranged at the reaction zone.

3. The method of claim 2, wherein the thin film is made of a material selected from the group consisting of nitrocellulose and paper.

4. The method of claim 1, wherein an enzyme-absorption hole is formed on the conducting platform.

5. The method of claim 1, capable of being applied in detecting a protein.

6. The method of claim 5, wherein the protein is selected from the group consisting of food genes, antibiotics, prisons, insecticide, HIV virus, SARS virus, and the likes.

7. The method of claim 1, wherein a diversion hole is arranged at the center of the reaction zone of the testing platform and is used for filtering/draining the excess testing antibody to an area underneath the reaction zone.

8. The method of claim 7, wherein the drained excess antibody is absorbed by a water absorbing polymer.

9. The method of claim 8, wherein the water absorbing polymer is a sponge.

10. An ELISA analytic apparatus, comprising:
a testing platform, having a reaction zone arranged thereon and used for enabling a specific antigen, a testing antibody, a bio-reagent and an enzyme to react with each other therein;
a conducting platform with an extending end, have a conducting circuit arranged at a side thereof while the side with the conducting circuit is adapted for attaching to the reaction zone of the testing platform; and
an evaluation instrument, connected to the extending end of the conducting platform for enabling the same to conduct an accurate measurement upon the reaction result of the reaction zone and thus acquiring a value of potential corresponding thereto.

11. The apparatus of claim 10, wherein a thin film is arranged at the reaction zone.

12. The apparatus of claim 11, wherein the thin film is made of a material selected from the group consisting of nitrocellulose and paper.

13. The apparatus of claim 10, wherein an enzyme-absorption hole is formed on the conducting platform.

14. The apparatus of claim 10, wherein a diversion hole is arranged at the center of the reaction zone of the testing platform and is used for filtering/draining the excess testing antibody to an area underneath the reaction zone.

15. The apparatus of claim 15, wherein the drained excess antibody is absorbed by a water absorbing polymer.

16. The apparatus of claim 15, wherein the water absorbing polymer is a sponge.

17. The apparatus of claim 10, wherein a plurality of bumps are arranged on the conducting platform, each being arranged at a position corresponding to a concave formed on the testing platform for adapting the conducting platform to attached upon the testing platform.

18. The apparatus of claim 10, wherein an adhesive is arranged on at least one object selected from the group consisting of the testing platform and the conducting platform for enabling the conducting platform to attach upon the testing platform.

19. The apparatus of claim 10, wherein a plurality of wedges are arranged on the conducting platform, each being arranged at a position corresponding to a recess formed on the testing platform for adapting the conducting platform to attached upon the testing platform as each wedge is pushed to jammed inside its corresponding recess.
